(19) United States
(12) Patent Application Publication
(10) Pub. No.: US 2011/0027899 A1
(43) Pub. Date: Feb. 3, 2011

(54) HAZARDOUS CHEMICALS DETECTOR & METHODS OF USE THEREOF

(76) Inventor: James M. Hargrove, Riverside, CA (US)

Correspondence Address: LOZA & LOZA LLP, 305 North Second Ave., #127, Upland, CA, 91786-6064, US

(21) Appl. No.: 12/703,582

(22) Filed: Feb. 10, 2010

Related U.S. Application Data
(60) Provisional application No. 61/151,320, filed on Feb. 10, 2009.

(51) Int. Cl. G01N 33/00

(52) U.S. Cl. 436/117; 422/83

(57) ABSTRACT

Embodiments of the invention are directed to an apparatus and method for detecting explosive compounds by air sampling followed by subjecting the air sample to a detection method. In one embodiment, a test area is sampled by drawing air from the vicinity of the test area, heating or irradiating the air sample and subjecting the irradiated sample to a detection method. With respect to nitrogen-containing explosive compounds, heating or irradiating the air sample produces nitrogen dioxide (NO₂). With respect to non-nitrogen-containing explosive compounds (e.g., oxygen-containing explosive compounds), the air sample may be exposed to a source of nitrogen monoxide (NO) to generate nitrogen dioxide (NO₂). With respect to nitrogen-containing samples that preferentially generate nitrogen monoxide (NO) rather than nitrogen dioxide (NO₂), gas titration may be integrated into the system to convert nitrogen monoxide (NO) to nitrogen dioxide (NO₂). The resultant nitrogen dioxide (NO₂) may be detected by a nitrogen dioxide analyzer (“NO₂-analyzer”) by a device such as, but not limited to, a cavity attenuated ring down spectrometer with gated integrated detection (CARDS-GID), a cavity phase shift spectroscopy (CAPS)-based instrument, or a laser-induced fluorescence detector (LIF).

### CLAIM OF PRIORITY

This application claims priority to U.S. App. Ser. No. 61/151,320 filed Feb. 10, 2009 and hereby incorporated by reference.

### FIELD

Various embodiments of the invention pertain to a detection method for detection of trace quantities of explosive components including nitrates, oxidizers and peroxides, or other radical-generating agents.

### BACKGROUND

An explosive material is a material that either is chemically or otherwise energetically unstable or produces a sudden expansion of the material usually accompanied by the production of heat and large changes in pressure (and typically also a flash and/or loud noise) upon ignition. Examples of explosive compounds include, but are not limited to ammonium nitrate, nitroglycerin, acetone peroxide, trinitrotoluene (TNT), nitrocellulose, RDX, PETN, and HMX. Recently, the detection of explosives in venues such as ports (which serve as the entry point for importing foreign commodities), airports and other border entries, has become extremely important in view of the global spread of terrorist attacks.

The detection of explosive compound residues has been attempted with mass spectroscopy, fluorescence detection, and nitric oxide detection. Mass spectroscopy requires expensive and cumbersome equipment that needs frequent maintenance. Fluorescence detection may not be sensitive enough for all applications, and requires that samples be accessible to sampling by wipes that are then irradiated to detect any residue. Nitrogen monoxide detection has been developed based on the principle that many explosives can be made to generate nitrogen monoxides. Conventional equipment used in these techniques is relatively insensitive, requires frequent calibration and service, and/or does not detect all of the possible types of explosives.

In high traffic areas such as airports, cargo terminals and ports, the presence of an explosive device can be difficult to detect. An explosive device can be hidden on an individual or within cargo filled with legal and traceable imported goods. Airports and cargo terminals often use X-ray and CAT equipment to rapidly scan large numbers of items (e.g., shipping boxes) in an attempt to identify items that could contain explosives. These methods require highly trained operators, and the quality of detection is dependent on the skill of the operator. If something suspicious is preliminarily detected, it is still not certain that it is a bomb until it is removed from inside the package and examined.

Consequently, a method, system or apparatus which cures the deficiencies as described previously is desirable.

### SUMMARY

A method for detecting explosive compounds, comprising: (a) collecting an air sample in a vicinity of an object; (b) subjecting the air sample to one of heat or irradiation to generate nitrogen dioxide (NO₂); and (c) measuring the generated nitrogen dioxide (NO₂) by a nitrogen dioxide detector is herein disclosed. For nitrogen-containing explosive compounds which preferentially decompose to nitrogen monoxide (NO), the method may further comprise (d) converting the generated nitrogen monoxide (NO) to nitrogen dioxide (NO₂) by gas phase titration. The gas phase titration may comprise: (i) exposing the air sample to ozone; and (ii) allowing the air sample to remain in a heated reaction chamber for a predetermined amount of time. For non-nitrogen-containing explosive compounds, the method may further comprise (e) adding nitrogen monoxide (NO) to the air sample stream during subjecting the air sample to one of heat or irradiation.

Collecting an air sample may comprise one of mechanically transporting a collected sample to an instrument, vacuum collection of vapor or particles, and vortex vacuum sampling. Subjecting the air sample to one of heat or irradiation may comprise heating the sample to between 150 degrees Celsius and 300 degrees Celsius. After collecting an air sample, the air sample may be introduced into a gas scrubber mechanism. Furthermore, after introducing the air sample into the gas scrubber mechanism, the air sample may be introduced into a cyclone. Furthermore, after introducing the air sample into the cyclone, the air sample may be introduced into a thermolysis heater. Furthermore, after introducing the air sample into the thermolysis heater, the air sample may be introduced into a nitrogen dioxide analyzer. The nitrogen dioxide analyzer may be one of a cavity attenuated ring down spectrometer with gated integrated detection (CARDS-GID), a cavity phase shift spectroscopy (CAPS)-based instrument, a cavity enhanced absorption analyzer (CEAS), or a laser-induced fluorescence detector (LIF). During subjecting the air sample, adding carbon monoxide (CO) or a hydrogen-containing organic compound to the sample stream may enhance the conversion of nitrogen monoxide (NO) to nitrogen dioxide (NO₂).

A system for detecting explosive compounds, comprising: (a) an inlet for taking in an air sample; (b) at least one filter mechanism in fluid communication with the inlet; (c) one of a heater or radiation device in fluid communication with the at least one filter mechanism; and (d) a nitrogen dioxide analyzer in communication with the heater or radiation device is herein disclosed. The system may further comprise (e) a gas titration system in fluid communication with the system for detecting explosive compounds, the gas titration system comprising: (f) an ozone generator; and (g) a heated reaction chamber in fluid communication with the ozone generator wherein the heated reaction chamber includes a plurality of glass beads.

The system may further comprise a nitrogen monoxide (NO) source in fluid communication with the system for detecting explosive compounds. The system may further comprise a carbon monoxide (CO) or a hydrogen-containing organic compound source in fluid communication with the system for detecting explosive compounds. The hydrogen-containing organic compound may be, e.g., isopropyl alcohol. The nitrogen dioxide analyzer may be one of a cavity attenuated ring down spectrometer with gated integrated detection (CARDS-GID), a cavity phase shift spectroscopy (CAPS)-based instrument, cavity enhanced absorption (CEAS) or a laser-induced fluorescence detector (LIF). The system may further comprise a cyclone device in fluid communication between the at least one filter mechanism and the heater or radiation device. The heater or radiation device may be a thermolysis heater.

### BRIEF DESCRIPTION OF THE DRAWINGS

FIG. 1 illustrates a block diagram cavity ring-down apparatus which may be used in an embodiment of the invention.

FIG. 2 illustrates a thermolysis heater which may be used in an embodiment of the invention.

FIG. 3 illustrates a system for measuring particle nitrates which may be used in an embodiment of the invention.

FIG. 4 illustrates a device for the conversion of NO to NO₂ which may be used in an embodiment of the invention.

FIG. 5 is the reaction scheme for amplifying NO₂ generation from a radical generating compound.

### DETAILED DESCRIPTION

In the following description numerous specific details are set forth in order to provide a thorough understanding of the invention. However, one skilled in the art would recognize that the invention might be practiced without these specific details. In other instances, well known methods, procedures, and/or components have not been described in detail so as not to unnecessarily obscure aspects of the invention.

Embodiments of the invention are directed to an apparatus and method for detecting explosive compounds by air sampling followed by subjecting the air sample to a detection method. In one embodiment, a test area (including, but not limited to, an area about a person or an object) is sampled by drawing air from the vicinity of the test area, heating or irradiating the air sample and subjecting the irradiated sample to a detection method. With respect to some nitrogen-containing explosive compounds, heating or irradiating the air sample produces nitrogen dioxide (NO₂). With respect to some non-nitrogen-containing explosive compounds (e.g., oxygen-containing explosive compounds), the air sample may be exposed to a source of nitrogen monoxide (NO) to generate nitrogen dioxide (NO₂). With respect to nitrogen-containing samples that preferentially generate nitrogen monoxide (NO) rather than nitrogen dioxide (NO₂), gas titration may be integrated into the system to convert nitrogen monoxide (NO) to nitrogen dioxide (NO₂). The resultant nitrogen dioxide (NO₂) may be detected by a nitrogen dioxide analyzer (“NO₂-analyzer”) by a device such as, but not limited to, a cavity attenuated ring down spectrometer with gated integrated detection (CARDS-GID), a cavity phase shift spectroscopy (CAPS)-based instrument, or a laser-induced fluorescence detector (LIF).

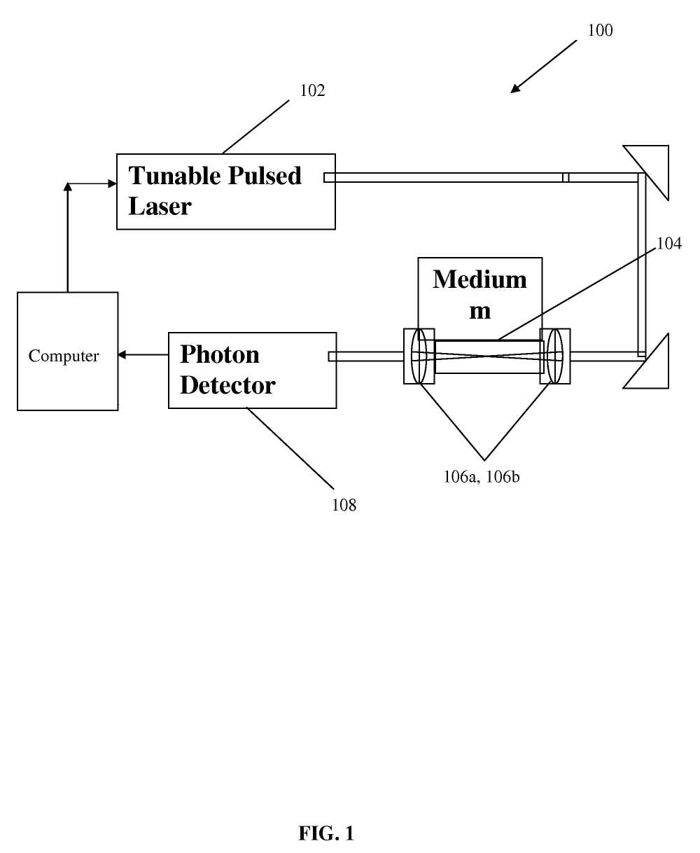

FIG. 1 illustrates a block diagram of a cavity ring-down apparatus 100 which may be used according to embodiments of the invention. Generally, a laser or other light source (such as a light-emitting diode (LED)) 102 illuminates a cavity 104 with minors 106a, 106b at each end. The light bounces back and forth between mirrors 106a, 106b hundreds or thousands of times enhancing absorption between the mirrors 106a, 106b. If a sample is passed between the minors, a change in light absorption occurs for a very small concentration of sample. Resultant wavelengths are measured by a photon detector 108. If a phase shift is measured, then it is termed cavity phase shift spectroscopy (CAPS). If the decay is measured by analog integration, then it is termed Cavity Attenuated Ring Down Spectroscopy-Gated Integrated Detection (CARDS-GID). See, Hargrove, J. M., The Application of Cavity Ring-Down Spectroscopy to Atmospheric and Physical Chemistry, University of California, Riverside, Riverside; and U.S. patent application Ser. No. 12/269,627 filed Nov. 12, 2008, which are hereby incorporated by reference. The system as described is referred to as a “nitrogen dioxide analyzer” or an “NO₂-analyzer.”

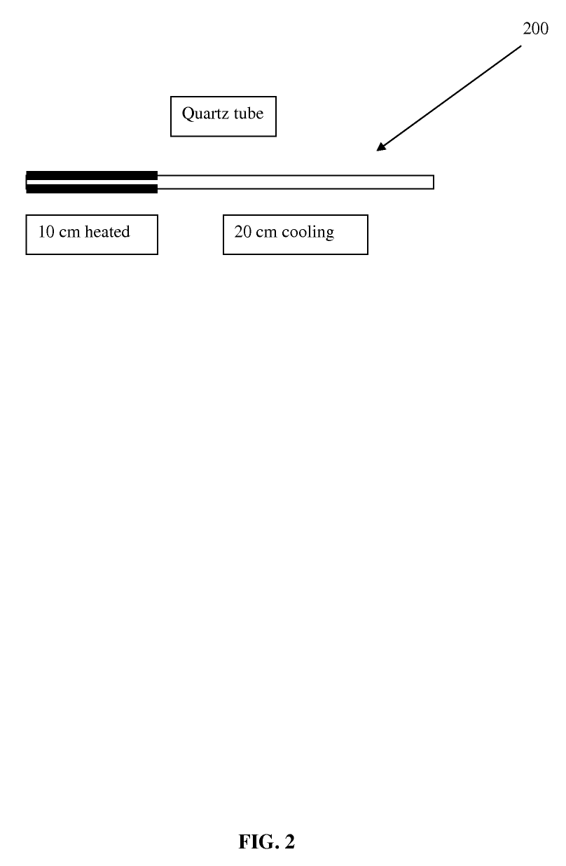

FIG. 2 illustrates a thermolysis heater 200 which may be used according to embodiments of the invention. See, Day, D. A., et al., A thermal dissociation laser-induced fluorescence instrument for in situ detection of NO₂, peroxy nitrates, alkyl nitrates and HNO₃, J. of Geophysical Research-Atmospheres, 107:D5-6 (2002), which is hereby incorporated by reference. In one embodiment, a thermolysis heater may cause an nitrogen-containing explosive compound (if present) to generate nitrogen dioxide (NO₂) that is in turn detected by an NO₂-analyzer. In another embodiment, radiation may be used to generate nitrogen dioxide (NO₂) from nitrogen-containing explosive compounds.

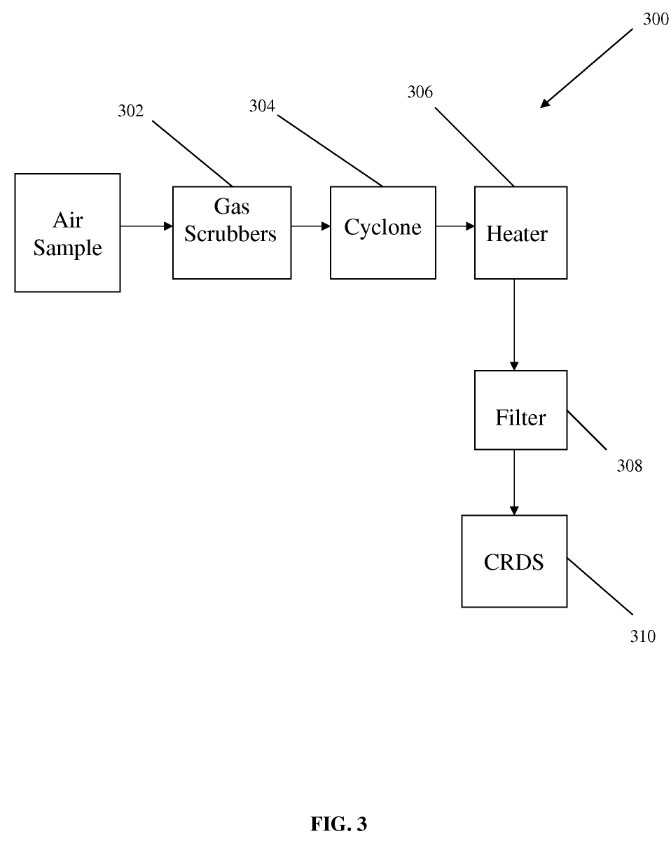

FIG. 3 illustrates a system 300 for measuring particle nitrates which may be used according to an embodiment of the invention. In one embodiment, an air sample is drawn from the vicinity of a test area. The test area may be, for example, the vicinity around a cargo box. Examples of air sampling methods include, but are not limited to, mechanically transporting a collected sample to an instrument, vacuum collection of vapor or particles, and vortex vacuum sampling as known by one of ordinary skill in the art. In one embodiment, the air sampling may be effectuated by a simple tube inlet or an enclosure that is flushed with air to draw out and mix the sample before reaching the inlet. After collection, the air sample may be introduced into carbon, silica or a combination thereof filter mechanisms 302 to remove, e.g., ambient nitrogen dioxide (NO₂) and water vapor.

Next, the air sample may be introduced into a cyclone 304 which works by creating a vortex where heavier particles, such as mineral dust, dirt, or pollen, strike the walls of cyclone 304 and fall into a cup (not shown) to be subsequently removed. Next, the air sample may be introduced into a thermolysis heater 306 (see FIG. 2) to generate nitrogen dioxide (NO₂) from any nitrogen-containing explosive compounds (if present). In some embodiments, the thermolysis may be set at a temperature in the range of between one-hundred and fifty (150) degrees Celsius and three hundred (300) degrees Celsius. Typically, the air sample is thereafter introduced into a TEFLON filter 308 to remove any unreacted particles. Finally, the sample may be introduced into a CRDS cavity 310 to detect the amount of nitrogen dioxide (NO₂), if any, which is proportional to nitrogen-containing explosive compounds if present in the air sample (see FIG. 1).

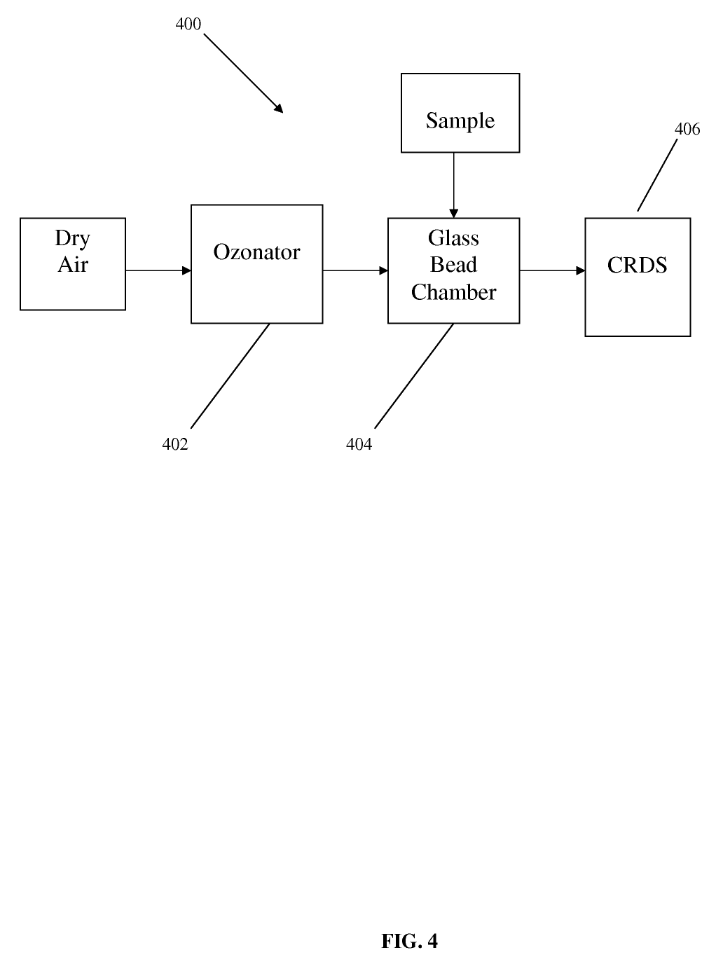

Because not all explosive compounds are nitrogen-containing, modifications to the system previously describe may be required. Oxidizers and peroxides are also components of explosives that may be desirable to detect. Such compounds may be detected by the same apparatus/method described previously by adding nitrogen dioxide (NO) to the sample stream and measuring the generated nitrogen dioxide (NO₂). For example, in one embodiment, the air sample may be exposed to a source of nitrogen monoxide (NO) so that the presence of non-nitrogen-containing explosive compounds, e.g., oxidizers or peroxides, can be detected by the reaction of such compounds with nitrogen monoxide (NO) to produce nitrogen dioxide (NO₂). The resultant nitrogen dioxide (NO₂), if any, is proportional to non-nitrogen-containing explosive particles, if present, and may be subsequently by detected by the NO₂-analyzer. In another embodiment, a radical chain-propagating species such as carbon monoxide (CO), isopropyl alcohol (C₃H₇OH), or any other suitable hydrocarbon may be added in addition to nitrogen monoxide (NO) to enhance the resulting signal of nitrogen dioxide (NO₂) (see FIG. 4).

Some explosive compounds, such as TNT, preferentially generate nitrogen monoxide (NO) (rather than nitrogen dioxide) and can then be detected by thermal generation of nitrogen monoxide (NO), conversion of nitrogen monoxide (NO) to nitrogen dioxide (NO₂) by gas phase titration, and detection of the resulting nitrogen dioxide (NO₂). FIG. 4 illustrates a device 400 for the conversion of nitrogen monoxide (NO) to nitrogen dioxide (NO₂) which may be used according to embodiments of the invention. As previously discussed, explosive compounds that decompose to produce nitrogen monoxide (NO) instead of nitrogen dioxide (NO₂), such as the nitroaromatics, may be detected by the conversion of the generated nitrogen monoxide (NO) to nitrogen dioxide (NO₂) by gas phase titration. As shown in FIG. 4, the sample from the test area is exposed to ozone from an ozone generator 402 and then spends approximately one minute in a reaction chamber 404 to allow nitrogen monoxide (NO) to be converted to nitrogen dioxide (NO₂) before introduction into the CRDS cavity 406 for detection of the resultant NO₂.

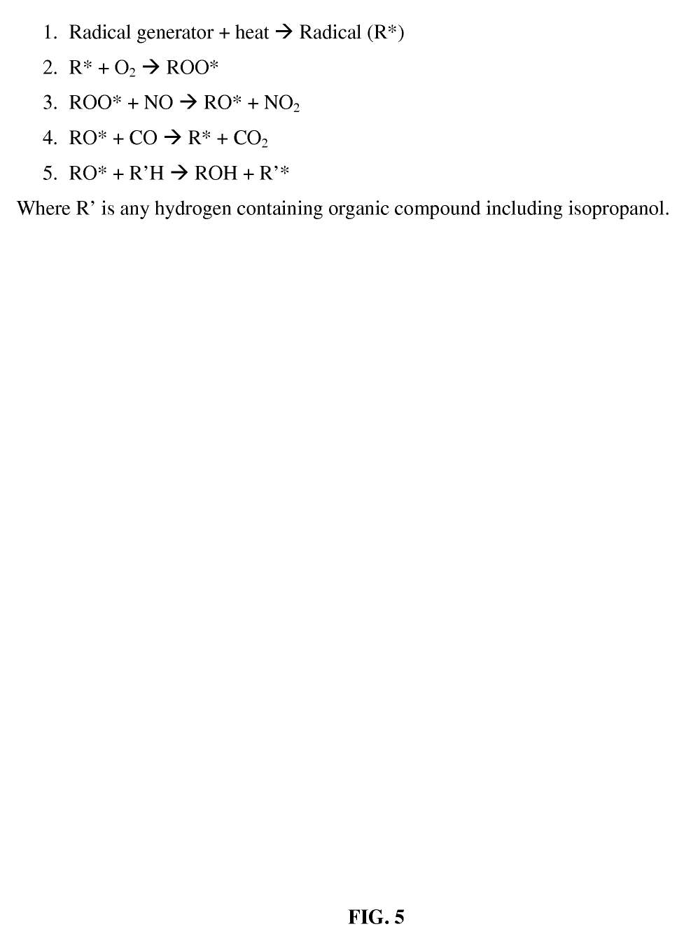

FIG. 5 is the reaction scheme for amplifying nitrogen dioxide (NO₂) generation from a radical generating compound.

Embodiments of the system as described previously have several advantages over conventional systems used to detect explosive materials. Unlike mass spectroscopy, the CRDS analyzer is simple and relatively inexpensive. CRDS does not need calibration or frequent maintenance. More types of explosives can be detected than with an NO₂-analyzer and the detection sensitivity is higher.

One or more of the components and functions illustrated in the figures may be rearranged and/or combined into a single component or embodied in several components without departing from the invention. Additional elements or components may also be added without departing from the invention. The apparatus, devices, and/or components illustrated in the figures may be configured to perform the methods, features, or steps illustrated in FIGS. 1-5.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications are possible. Those skilled, in the art will appreciate that various adaptations and modifications of the just described preferred embodiment can be configured without departing from the scope and spirit of the invention. Therefore, it is to be understood that, within the scope of the appended claims, the invention may be practiced other than as specifically described herein.

What is claimed is:

1. A method for detecting explosive compounds, comprising:
collecting an air sample in a vicinity of an object;
subjecting the air sample to one of heat or irradiation to generate nitrogen dioxide (NO₂); and
measuring the generated nitrogen dioxide (NO₂) by a nitrogen dioxide detector.

2. The method of claim 1, further comprising, for nitrogen-containing explosive compounds which preferentially decompose to nitrogen monoxide (NO), converting the generated nitrogen monoxide (NO) to nitrogen dioxide (NO₂) by gas phase titration.

3. The method of claim 2 wherein gas phase titration comprises:
exposing the air sample to ozone; and
allowing the air sample to remain in a heated reaction chamber for a predetermined amount of time.

4. The method of claim 1, further comprising, for non-nitrogen-containing explosive compounds, adding nitrogen monoxide (NO) to the air sample stream during subjecting the air sample to one of heat or irradiation.

5. The method of claim 1 wherein collecting an air sample comprises one of mechanically transporting a collected sample to an instrument, vacuum collection of vapor or particles, and vortex vacuum sampling.

6. The method of claim 1 wherein subjecting the air sample to one of heat or irradiation comprises heating the sample to between 150 degrees Celsius and 300 degrees Celsius.

7. The method of claim 1 wherein, after collecting an air sample, the air sample is introduced into a gas scrubber mechanism.

8. The method of claim 7 wherein, after introducing the air sample into the gas scrubber mechanism, the air sample is introduced into a cyclone.

9. The method of claim 8 wherein, after introducing the air sample into the cyclone, the air sample is introduced into a thermolysis heater.

10. The method of claim 9 wherein, after introducing the air sample into the thermolysis heater, the air sample is introduced into a nitrogen dioxide analyzer.

11. The method of claim 10 wherein the nitrogen dioxide analyzer is one of a cavity attenuated ring down spectrometer with gated integrated detection (CARDS-GID), a cavity phase shift spectroscopy (CAPS)-based instrument, a cavity enhanced absorption analyzer (CEAS), or a laser-induced fluorescence detector (LIF).

12. The method of claim 1 wherein, during subjecting the air sample, adding carbon monoxide (CO) or a hydrogen-containing organic compound to the sample stream to enhance the conversion of nitrogen monoxide (NO) to nitrogen dioxide (NO₂).

13. A system for detecting explosive compounds, comprising:
an inlet for taking in an air sample;
at least one filter mechanism in fluid communication with the inlet;
one of a heater or radiation device in fluid communication with the at least one filter mechanism; and
a nitrogen dioxide analyzer in communication with the heater or radiation device.

14. The system of claim 13, further comprising:
a gas titration system in fluid communication with the system for detecting explosive compounds, the gas titration system comprising:
an ozone generator; and
a heated reaction chamber in fluid communication with the ozone generator wherein the heated reaction chamber includes a plurality of glass beads.

15. The system of claim 13, further comprising, a nitrogen monoxide (NO) source in fluid communication with the system for detecting explosive compounds.

16. The system of claim 13, further comprising, a carbon monoxide (CO) or a hydrogen-containing organic compound source in fluid communication with the system for detecting explosive compounds.

17. The system of claim 16 wherein the hydrogen-containing organic compound is isopropyl alcohol.

18. The system of claim 13 wherein the nitrogen dioxide analyzer is one of a cavity attenuated ring down spectrometer with gated integrated detection (CARDS-GID), a cavity phase shift spectroscopy (CAPS)-based instrument, cavity enhanced absorption (CEAS) or a laser-induced fluorescence detector (LIF).

19. The system of claim 13, further comprising:
a cyclone device in fluid communication between the at least one filter mechanism and the heater or radiation device.

20. The system of claim 13 wherein the heater or radiation device is a thermolysis heater.
